(19) United States
(12) Patent Application Publication
(10) Pub. No.: US 2010/0028417 A1
(43) Pub. Date: Feb. 4, 2010

(54) USE OF SUBSTITUTED GLYCERIN DERIVATIVES FOR PRODUCING A PHARMACEUTICAL PREPARATION

(76) Inventors: Dieter Mueller-Enoch, Dornstadt (DE); Thomas Haehner, Dornstadt (DE)

Correspondence Address: Briscoe, Kurt G.;Norris McLaughlin & Marcus, PA, 875 Third Avenue, 8th Floor, New York, NY, 10022, US

(21) Appl. No.: 12/298,174

(22) PCT Filed: Apr. 27, 2007
(86) PCT No.: PCT/DE2007/000767

§ 371 Date: Mar. 30, 2009

(30) Foreign Application Priority Data
Apr. 28, 2006 (DE) 10 2006 019 907.3

(51) Int. Cl. A61K 31/4178; A61K 9/127; A61P 35/00; A61P 3/06

(52) U.S. Cl. 424/450; 514/397

(57) ABSTRACT

A compound of the formula (1) or pharmaceutically acceptable salts thereof can be used for producing a pharmaceutical preparation for preventing or treating cancerous diseases, pathological sequelae of alcohol abuse, viral hepatitis, steatohepatitis, acute and chronic pancreatitis, toxic renal disorders, hepatic insulin resistance in diabetes mellitus, liver damage associated with Wilson's disease and/or sideroses, ischemic reperfusion damage, for use as an antidote to environmental toxins and prescription drug intoxication, for prolonging the retention time of drugs in the organism, and/or for combating toxic side effects on administration of chemotherapeutic agents. B₆, B₇ and B₈ are identical or different and denote O, S, NH, PO₄, Se, SO₄. R₁ is identical to H or a C₆ and/or C₇ to C₂₆ and/or C₂₀ alkyl chain; and R₂, R₃, R₄ and R₅ can be identical or different and denote an H or a C₁ to C₃ alkyl, alkanol, alkylamine and/or alkyl thiol group. R₆, R₇ and R₈ can be identical or different; and H can denote a substituted or unsubstituted C₆ and/or C₇ to C₂₆ alkyl radical, a glycoside radical, a positively or negatively charged amino acid radical, a —(CH₂)ₙ—N⁺ (R₉, R₁₀, R₁₁), where R₉, R₁₀, R₁₁ is H, methyl, ethyl and/or propyl radical, and where at least two of R₂, R₃, R₄, and R₅ can together form a polyol radical, and n denotes a whole number from 1 to 5.

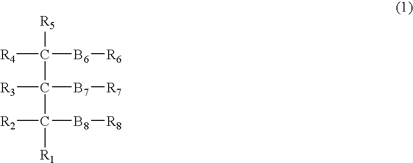

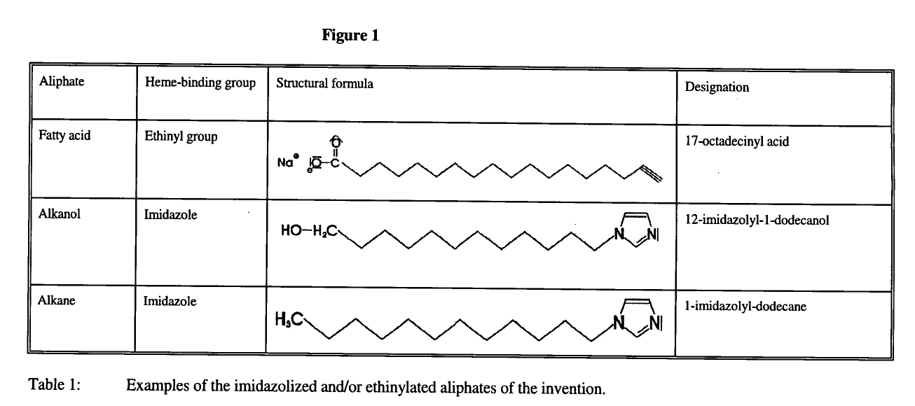

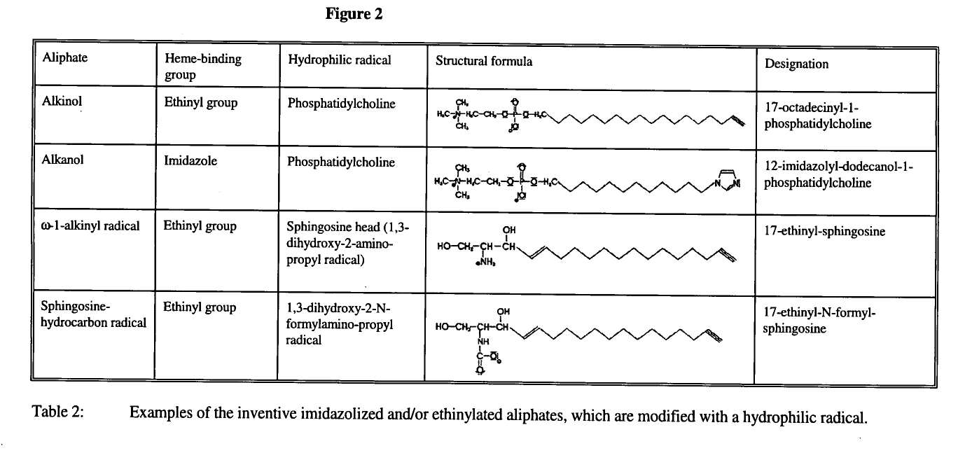

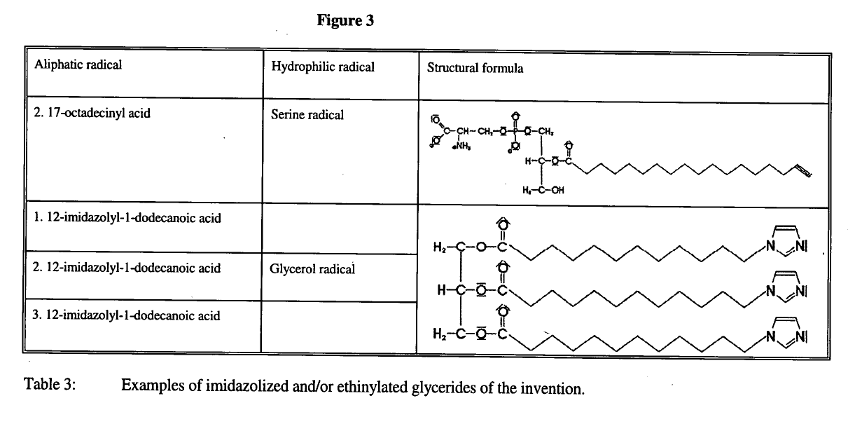

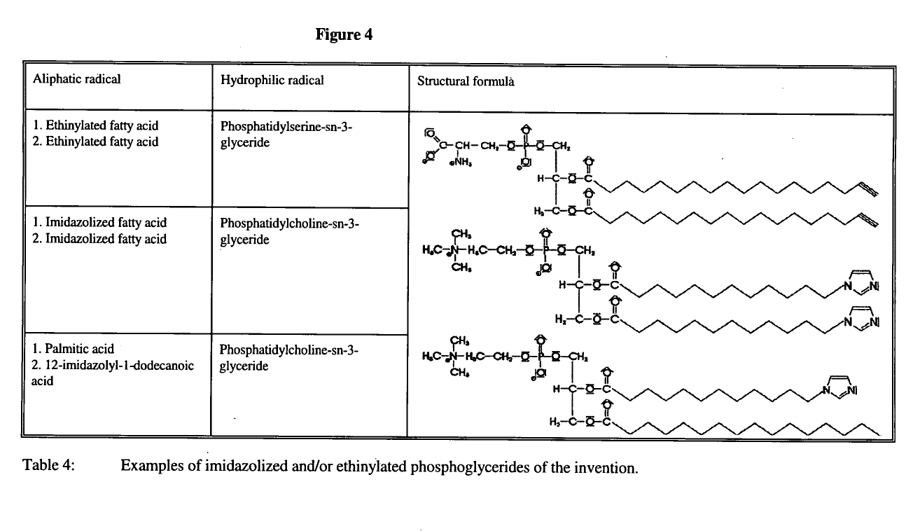

The invention relates to the use of substituted glycerol derivatives or the pharmaceutically acceptable salts of these compounds for producing a pharmaceutical preparation. Moreover, the invention relates to such preparations themselves, in particular for their application in humans.

Excessive alcohol consumption for a prolonged period of time frequently leads to a liver disease—the so-called fatty liver—which can further develop into an inflammation of the liver or rather hepatitis and to a cirrhosis of the liver in the late stage. Hence, the risk and the degree of the respective liver damage is a direct function of the amount and the duration of the alcohol consumption, so that the risk varies from individual to individual. An alcohol induced inflammation of the liver (alcohol hepatitis) is a disease that may be life threatening under some circumstances and may be accompanied by fever, jaundice as well as an increase in the white blood cells. Such alcohol induced inflammations of the liver are curable by total abstinence of alcohol, except for scars in the case of cirrhosis of the liver.

Besides this alcohol-induced so-called fatty liver hepatitis or alcoholic steatohepatitis (ASH), hepatitises also develop in persons, who do not indulge in alcohol abuse or do not consume any alcohol at all. Such hepatitises are induced, for example, by environmental toxins, for example, when working in painting plants and/or also induced by prescription drugs.

Alzheimer's disease is a progressive dementia, which ultimately leads to the complete loss of memory and personality. It is induced by protein deposits in the nerve cells—the plaques—which are composed of β-amyloid and/or the so-called τ-proteins. To date the actual cause is unknown, although not only metabolic disorders and gene mutations but also so-called slow viral infections and/or prions are being discussed.

In the case of Alzheimer's disease it is known that a lipid oxidation in the brain of transgenic mice triggers the formation of plaque. This lipid oxidation leads to a G-amyloid precursor protein, which forms the well-known plaques.

Parkinson's disease is a degeneration of the dopaminergic neurons in the substantia nigra of the brain region. It concerns the most frequent neurological disorder in old age. The early signs are, in particular, trembling movements (tremors), a fundamental mood of depression, apathy as well as retarded thinking processes. In this case, too, it is suspected that reactive oxygen species are involved in the development of the disorder.

It is known that oxidation processes in the metabolic process take place with the aid of cytochromes. Cytochromes are a plurality of different enzymes, the active center of which exhibits a heme structure. It catalyzes the transfer of electrons to an acceptor in a plurality of oxidation and hydroxylation reactions.

For example, the cytochromes of the P450 family (CYP 450) play an important role. In this case they are monooxygenases, which are ubiquitous and belong to the most important enzymes in the metabolism of hydrophobic exogenous substances and in the modification of hydrophobic hormones—the steroids.

One of the main tasks of the cytochrome P450 enzymes is to solubilize exogenous substances by hydroxylation and in this way to deliver them to the renal excretion. Therefore, the cytochrome P450 enzymes play an important role in the detoxification process.

It is estimated that approximately half of all current drugs are hydroxylated by the cytochrome P450 enzymes of the liver. Therefore, the retention time of many drugs in the body is significantly reduced to some extent by the activity of the cytochrome P450 enzymes. In mammals the predominant amount of cytochrome P450 is found in the liver, because the liver is the central detoxifying organ. The cytochrome P450 is usually present in the combined state on the membrane of the endoplasmic reticulum.

Cytochrome P450 enzymes also play a key role in promoting the resistance of insects to insecticides and the resistance of plants to herbicides.

In its basic structure cytochrome P450 exhibits a six coordinated heme group, where a reaction of the following structure

RH+O₂+2H⁺+2e⁻→ROH+H₂O

is catalyzed. At the same time, the two electrons, which are necessary for this reaction, are made available—for example, by NADPH cytochrome P450 reductases, which are associated with the enzyme complex. In this way cytotoxic, reactive oxygen species (ROS) are produced, inter alia, at P450.

The cytochrome P450 itself is present in a variety of different forms, like 1A1, 2B1, 2C9, 2J2, 2E1, 3A1, etc. Thus, for example, approximately 60% of the difference between the cytochrome P450 2B1 in rats and the human cytochrome P450 2E1 lies in their amino acid sequence. That is, both the structures of the active and catalytic centers as well as the size and shape of the access channels for the substrate are drastically different. In the end the result is that both enzymes metabolize totally different classes of substrates. The isoform 2E1 reacts with considerably smaller molecules—for example, ethanol, acetone or also p-nitrophenol.

This variance is almost a common characteristic of all isoforms of P450 heme proteins. That is, it is not possible to draw a conclusion about one isoform from another isoform. Therefore, knowledge, acquired with the isoform 2B1 in rats, cannot be transferred to the 2E1 isoform in humans.

Moreover, polymorphisms allow individual variances in the function of a given cytochrome P450 form to occur even inter-specifically. This is the reason for the very wide variation in the intensity and duration of the effects and side effects from patient to patient given the same dose of a drug.

It is known that both alcohol consumption and non-alcoholic fatty liver hepatitis and pancreatitis induce the synthesis of the cytochrome P450 2E1. The function and mechanism of action of this isoform, which is much different from other cytochromes, is described, for example, by M. H. Wang et al. in Archives of Biochemistry and Biophysics, (1995), Vol. 317, pages 299 to 304. According to this article, the enzyme exhibits an approximately 15 Å long duct, at the end of which is the reactive center with a heme ring exhibiting a central iron atom.

For a long time it has been suspected that even chemotherapeutic agents, such as those used in the treatment of cancer, are decomposed by the cytochrome P450 enzymes.

However, a recent article by Jiang et al. (“Cytochrome P450 2J2 Promotes the Neoplastic Phenotype of Carcinoma Cells and is Up-regulated in Human Tumors” in Cancer Res. 2005, 65: 4707-4715) revealed for the first time that the cytochrome P450 can even have a cancer promoting effect.

It was demonstrated that the gene expression of cytochrome P450 2J2 is up-regulated in human tumors. The cytochrome P450 2J2 is an epoxygenase, which converts the substrate arachidonic acid into four different isomeric epoxyeicosatrienoic acids (EETs). Furthermore, the study showed that the EETs exhibit an apoptosis-inhibiting effect, because they protect the tumor cells against the effect of the tumor necrosis factors, and in this way increase the lifespan of the cancer cells. Moreover, they promote the mitosis as well as the proliferation of tumor cells.

Similarly it could be demonstrated that the EETs promote the angiogenesis—that is, the formation of new blood vessels. This process plays an important role in the growth of tumors (Pozzi A. et al. “Characterization of 5,6 and 8,9 Epoxyeicosatrienoic Acids (5,6 and 8,9 EET) as Potent in Vivo Angiogenic Lipids,” J. Biol. Chem., Vol. 280, pp. 27138-27146, 2005).

In contrast, the article by Schattenberg et al. (“Hepatocyte CYP2E1 overexpression and steatohepatitis lead to impaired hepatic insulin signaling” in J. Biol. Chem. 2005; Vol. 280, pp. 9887-9894) links for the first time an overexpression of the cytochrome P450 with diabetes.

Müller-Enoch et al. describe in Z. Naturforsch. (2001) 56c, pages 1082-1090, the inhibiting of the cytochrome P450 2B1 in rats by means of lysophosphatidylcholines, lysophosphatidylinositol as well as arachidonic and oleinic acids and/or by monoacylglycerols, monooleylglycerols, and monopalmitoylglycerols.

Furthermore, T. Haehner, D. Müller-Enoch et al. in Z. Naturforschung (2004) 59c, pages 599-605, describe the influence of single chain lipid molecules on the activity of the isoform cytochrome P450 2B1 in rats.

The object of the invention is to provide means for producing a pharmaceutical preparation, which is suitable for preventing or treating cancerous diseases, pathological sequelae of alcohol abuse, viral hepatitis, steatohepatitis, acute and chronic pancreatitis, Alzheimer's disease, Parkinson's disease, toxic renal failure, diabetes mellitus, Wilson's disease, sideroses, ischemic reperfusion damage, and/or arteriosclerosis, for use as an antidote to environmental toxins and prescription drug intoxication, for prolonging the retention time of drugs in the organism, or for combating toxic side effects on administration of chemotherapeutic agents.

This object is achieved with a compound having the features defined in the claims.

In particular, it was found surprisingly that the aforementioned diseases can be treated with such compounds. These compounds inhibit the formation of reactive oxygen species (ROS), in particular, the oxygen radicals, like the superoxidant (O₂.⁻) as well as the hydroxyl radical (.OH), which are not consumed in a direct redox reaction, at the cytochrome P450, in particular, at the isoforms of the group 2, especially 2E1, as well as 2J2.

The inventive compounds exhibit the formula

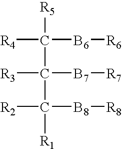

Moreover, the invention may also relate to the pharmaceutically acceptable salts of these compounds.

Basically the radicals R₁/R₆/R₇/R₈ may assume the form R—X. In the formula R stands for an aliphatic or aromatic hydrocarbon radical, which has preferably 6 to 40 carbon atoms and exhibits, in particular, a terminal hydrophilic radical A, and X stands for a radical, exhibiting at least one free electron pair of a carbon or heteroatom and/or π electrons. The radical R is, in particular, lipophilic.

Usually the radical R is an alkyl radical. Thus, it may be straight chained or branched, exhibit single bonds, double bonds or triple bonds and may be substituted. Usually it exhibits an aliphatic backbone having 6 to 26, in particular 8 to 22 carbon atoms. Practical are hydrocarbon chains having a backbone of 10 to 15, in particular 10 to 13 carbon atoms. If R is an alicyclic or aromatic hydrocarbon radical, which may be condensed and/or may be substituted lipophilically, then it usually exhibits at least 5 and/or 6 and at most 40 and/or at most 25 carbon atoms. Other practical minimum lengths are 7 and/or 8 C atoms; and other practical maximum lengths are 22 and/or 20 C atoms.

Practical radicals X are heterocycles as well as alkinyl radicals. The heterocycles are heterocycles, which contain, in particular, nitrogen, oxygen and/or sulfur, The heterocycles may be aromatic and/or non-aromatic and usually exhibit 5 or 6 ring atoms. In appropriate cases X may also be a condensed heterocycle. For example, such heterocycles are imidazole, pyrrole, pyrazole, pyridine, pyrazine, indole, isoindole, indazole. Preferred heterocycles are rings, which exhibit 6 and particularly 5 atoms and have one, two or three heteroatoms. Additional suitable heterocycles are, for example, thiazoles, triazoles, furans.

Preferred alkynes exhibit the structure —C≡C—R₁₂, where R₁₂ is a hydrogen or an optionally substituted C₁ to C₁₅ and/or maximally C₁₀ alkyl radical, which in turn may exhibit optionally double or triple bonds. Usually, however, R₁₂ exhibits maximally 5, in particular maximally 3 C atoms. In an additional practical embodiment of the invention, the radical X denotes, for example,
primary, secondary and tertiary amines,
substituted or unsubstituted diazo functions, such as hydrazines and hydrazones,
nitrile, isonitrile,
S-containing functional groups, such as thiocyanates and isothiocyanates, alkyl sulfides, sulfoxides, thiol groups,
methylene dioxy function,
alkyl ether and alkyl thio ether.

The radicals X are expediently radicals, which coordinate with the prosthetic heme group.

In a preferred embodiment R₁, which usually denotes a hydrogen or a C₆ and/or C₇ to C₂₆ and/or C₂₀ alkyl chain, exhibits one or more double and/or triple bonds. Similarly the hydrocarbon backbone can be formed with alicyclic and/or aromatic hydrocarbons, where, in this case owing to the ring structures, up to 40 carbon atoms may be necessary. In an especially preferred embodiment R₁ exhibits an α-terminal double bond and an ω-terminal triple bond, such as ω-acetylenic sphingosine. In principle, it is also possible to arrange the triple bond in a long molecule in the center in such a manner that it is coordinated with the heme, as, for example, in the case of eicosatrienoic acids or ω-acetylenated sphingosine. In another preferred embodiment the terminal triple bond may be substituted with a heterocycle or another heme-coordinated group. In principle, R₁ can exhibit one or more substituents. If R₁ contains a terminal heterocycle, then this heterocycle can exhibit one of the definitions, cited for R₆ to R₈.

The radicals R₂ to R₅ can be identical or different and are usually a C₁ to C₈ alkyl radical or hydrogen. The alkyl radicals may be optionally substituted and may be, for example, an alkanol, an alkylamine or an alkyl thiol radical. Especially preferred are hydrogen, methyl radicals, ethyl radicals and propyl radicals and/or their derivatives.

R₆ to R₈ are connected to the respective glycerol C atom by the bond B₆, B₇, and/or B₈ and can be identical or different and can be hydrogen, a C₆ and/or C₇ to C₂₆ and/or C₂₀, in particular C₈ and/or C₉ to C₂₂ and/or C₁₋₈ alkyl radical. These alkyl radicals can be both substituted and unsubstituted. Preferably such substituents are preferred that exhibit between the C₄ and/or C₉ to C₁₅ and/or C₁₄C atoms one or more triple bonds. Similarly the hydrocarbon backbone can be formed with alicyclic and/or aromatic hydrocarbons, so that in this case the ring structures may necessitate up to 40 and/or up to 25 carbon atoms.

It has been demonstrated that the inventive compounds, where at least one of the radicals R₁, R₆, R₇, and/or R₈ exhibits a heme-coordinated hydrocarbon backbone, such as 17-octadecinyl-1-acid, has proven to be especially effective in vitro, the retention time of these substances in the blood can be significantly prolonged, if the carboxy terminus of such a molecule is replaced, for example, with a sulfate radical, or a C atom, which is located adjacent to the carboxy terminus and belongs to the aliphatic backbone, is substituted through the addition of 2 methyl groups or through the addition of an aliphatic or aromatic ring. In this way even the in vivo activity is improved in conformity with the in vitro activity.

Thus, for example, 2,2-dimethyl-11-dodecinyl acid exhibits, like 10-undecinyl acid, in vitro a comparably high inhibition of cytochrome P450 activity, whereas it is far superior in vivo to the latter.

Preferably it is provided at the same time that the length of the aliphatic backbone comprises 6 and/or 9 to 26 and/or 13 carbon atoms, when R1 is an imidazole radical. One representative of this preferred group is, for example, the 12-imidazolyl-dedecanol or the 1-imidazolyl-dedecane. With respect to the structural formulas of these and other substances reference is made to the attached tables.

Furthermore, it is provided preferably that the length of the aliphatic backbone comprises 6 and/or 14 to 26 and/or 18 carbon atoms, if R1 is an ethinyl radical. One representative of this preferred group is, for example, 17-octadecinyl-1-acid.

Similarly the length of the aliphatic backbone comprises preferably 9 to 13 carbon atoms, if R1 is an ethinyl radical. Representatives of this preferred group are, for example, 2,2-dimethyl-11-dodecinyl acid, 10-undecinyl-sulfate, 10-undecinyl acid or 10-undecinol.

In another preferred embodiment at least one of the radicals R₆ to R₈ is a glycoside, in particular a monosaccharide, which may or may not be substituted, for example, with a sulfate or an amino radical. In the individual embodiments disaccharides or oligosaccharides are also very suitable for the inventive application purpose. Preferred saccharides are pentoses and hexoses as well as mixed saccharides. In principle, the glycosides may also be thio sugar.

In an especially preferred embodiment at least one of the R₆ to R₈ is a positively or negatively charged amino acid radical, in particular an amino acid with an additional amino group, where the amino acid groups of the structure —(CH₂)ₙ—N⁺ (R₉-R₁₁) denote an expedient alternative configuration. In this case n is usually a whole number between 1 and 5, where 1 to 3, in particular 2 is preferred. The radicals R₉ to R₁₁ are usually hydrogen or a methyl, ethyl or propyl group. In this case R₉ to R₁₁, may be identical or different. In a very preferred embodiment one of the X with one of the R₆ to R₈ radicals form together a phosphocholine group or another phosphate ester, such as phosphatidylethanolamine, phosphatidylserine or phosphatidylinositol radicals.

In a practical embodiment of the invention, the heme-coordinated group is an imidazole radical, which is bonded via a nitrogen atom, or an ethinyl radical (—C≡CR₁₂), where R₁₂ is hydrogen or a substituted or unsubstituted aliphatic C₁ to C₁₂ hydrocarbon radical.

The inventive application makes it possible to reduce the formation of such reactive oxygen species and to treat the aforementioned diseases.

In addition, it is possible to suppress or retard the metabolism-resulting hydroxylation of exogenous substances, in particular prescription drugs. In this way the retention time of these substances in the body is prolonged and/or the toxic side effects are reduced and/or even totally prevented. This feature is very important, for example, in the case of chemotherapies. One example is the effect of the platinums.

Moreover, these compounds offer the possibility of inhibiting the formation and proliferation of tumor tissues, since the application of the compounds, which are used according to the invention, suppresses the conversion of arachidonic acid into the proliferation-promoting and apoposis-impeding epoxyeicosatrienoic acids.

At this point it has been demonstrated that the biocompatibility of the said compounds, in particular those that are sparingly soluble under physiological conditions and/or owing to specific properties can pass only slightly through cell membranes and, therefore, do not adequately reach the site of action, can be enhanced by additional techniques described below. This also applies to such compounds that are rapidly broken down by the body's own enzymatic activity or are readily excreted via the renal excretion.

Therefore, some of the compounds, which are used according to the invention and that contain a heme-coordinated hydrocarbon backbone, are quickly resorbed by the muscle cells or fat cells on administration, so that only a very small amount reaches the site of action, for which reason higher doses of these compounds have to be administered.

For this reason another design of the invention envisages a further development of the inventive compounds. In this case the functional groups of the hydrocarbon radicals R₁, R₆, R₇, R₈, thus the terminal hydrocarbon radicals, the alcoholic OH group, the sulfate or the Co-A group or the organic acid group are modified by the addition of an additional hydrophilic radical.

The compounds, which are used according to the invention and which contain the above described heme-coordinated hydrocarbon radical, comprise compounds having the basic structure of the sphingosines, monoglycerides, diglyercides, and triglycerides as well as imidazolized or ethinylated phosphoglycerides, glycolipids, sphingolipids, gangliosides and cerebrosides, in particular their imidazolized or ethinylated forms.

The important feature in this case is that the hydrophilic radical does not have a negative impact on the bonding of the molecule to the active center of the cytochrome P450. This property can be accurately controlled through the choice of the length of the hydrocarbon backbone.

In another alternative embodiment, one of the R₁, R₆, R₇, R₈ is a choline or an ethanolamine, an α-, β-, or γ-hydroxyamino acid, such as serine, threonine, inositol or also galactoses.

B₆, B₇ and B₈ can be identical or different and denote O, S, NH, PO₄, Se, SO₄. The bond B_{6,7,8}, which links the respective carbon atom of the glycerol part and/or the polyol part R_{6,7,8}, is usually an ether bond and/or an ester bond between an alcoholic polyol and/or glycerol and an organic and/or inorganic acid group of R_{6,7,8}, such as a —C—O—C(O)—R_{6,7,8} group or a —C—O—P(O)—O—R_{6,7,8} group.

Examples of such compounds are, for example, 12-imidazolyl-dodecanol-1-phosphatidylcholine, 10-imidazolyl-decanol-1-phosphatidylcholine or 17-octadecinyl-1-phosphatidylcholine.

The compounds, which are used according to the invention, comprise, in particular, monoglycerides, diglycerides, or triglycerides, phospholipids and glycolipids.

The inventive application has the advantage that the compounds are not directly accessible to enzymes of the 1-oxidation metabolic process and are, therefore, not immediately metabolized.

It has been demonstrated that the phosphoglycerides and the triglycerides, which are used in an inventive embodiment and which are substituted with a radical R₁, R₆, R₇, R₈, in particular with such radicals, which exhibit a heme-coordinated hydrocarbon radical, at one or more sites of the glycerol radical, are transported to the sound organs and tumors without significant decomposition. It is suspected that following resorption these compounds are hydrolyzed in such a manner that one hydrocarbon radical or a plurality of hydrocarbon radicals is/are split off. The results are, inter alia, heme-coordinated monoglycerides, which are also called lysolipids. Said monoglycerides form with the aid of lipoproteins, thus non-covalent aggregates composed of lipids and proteins, the micelle-like particles and serve to transport water-insoluble lipids in the blood.

The same also applies, moreover, to heme-coordinated monoglyercides, such as the ethinylated and/or imidazolized monoglycerides (according to the above definition) that were already administered as such.

Since specific pathogenic tissues, such as tumors, have a high energy turnover and promote their own vascularization by releasing growth factors (VEGF, PDGF), the lipoproteins, loaded with the said heme-coordinated monoglycerides, migrate with the blood stream preferably into these tissues. Thus, the “packaging” of heme-coordinated hydrocarbon radicals in the form of lysolipids makes it possible to convey specifically said lysolipids into the said target organs.

As stated above, the heme-coordinated compounds exhibit the property that they interact with the active center of the cytochrome P450 and, in so doing, suppress its activity.

Against this background, the compounds must be attributed a potential role in the treatment of cancer. It can be expected that the administration of these compounds will inhibit the conversion of arachidonic acid into epoxyeicosatrienoic acids, said conversion being promoted by the cytochrome P450. The latter promote, as stated above, the cell division and proliferation and inhibit the apoptosis. Similarly it is expected that the administration of such compounds will inhibit the hydroxylation of chemotherapeutic agents, said hydroxylation ultimately leading to the excretion of said chemotherapeutic agents. Hence, such a compound could be used for a direct as well as for an adjuvant tumor therapy. For this reason the aforementioned preferred embodiment, which makes possible a targeted transport into sound organs, promises to be especially successful.

In these cases it is also important that the hydrophilic radical R₂ does not have a negative impact on the bonding of the molecule to the active center of the cytochrome P450. This state can be accurately controlled through the selection of the length of the hydrocarbon backbone.

Furthermore, the invention provides a pharmaceutical preparation, containing an inventive compound in a pharmaceutically acceptable carrier.

In addition, possible indications for an inventive compound and/or its pharmaceutical preparation lie in the treatment of the sequelae of alcohol abuse. They are, in particular, liver damage and also other alcohol induced inflammatory processes. In addition to the liver damage that is simply alcohol induced, nutrition-induced and endocrine factors, such as obesity as well as diabetes mellitus and hyperlipidemia, also cause, independently of alcohol, serious liver damage, which may range over fatty liver hepatitis (non-alcoholic steatohepatitis=NASH) as far as up to and including cirrhosis of the liver. Such alcoholic and non-alcoholic fatty liver diseases are often accompanied by a viral infection of the liver. In this case the consequence may be a very fast progression of the disease. It has been demonstrated that this should also be attributed, for example, to a synergistic production of reactive oxygen species (ROS) and the associated cell damage. All of the aforementioned diseases and/or their causes or their sequelae are treatable with the inventive compounds, which result in the inhibition of the cytochrome P450 activity.

It has also been found that these substances are quite appropriate for treating inflammations of the pancreas. Such inflammations and/or pancreatitis may be induced not only by alcohol abuse but also by toxic substances. They include, in particular, environmental toxins, like occupational chemicals or also prescription drugs. Even viral infections or endocrine factors of a metabolic origin may cause such inflammations of the pancreas. In all cases reactive oxygen species are involved in the development of the disease and in the progression of the disease.

The inventive pharmaceutical preparation has also proven to be appropriate for the treatment of diabetes mellitus—both type 1 and type 2 diabetes mellitus. In particular, it has been demonstrated that β-islet cells of the islands of Langerhans are especially sensitive to oxidative processes and that as the oxidative stress increases, these cells rapidly decrease. This oxidative stress can be avoided with the inventive pharmaceutical preparation, or at least drastically reduced.

The inventive pharmaceutical preparation has also proved to be effective in the treatment of Alzheimer's disease and Parkinson's disease. In this case it has been demonstrated, for example, that the inventive substances allow the concentration of dopamine to increase on account of decreased decomposition.

Even toxic renal disorders as well as other disorders, such as those induced by the side effects on the administration of chemotherapeutic agents, in particular cytotoxins, like metal complexes like cisplatinum, carboplatinum, titanocendichloride or gold complexes, are to be treated with the inventive drug. In this respect it has been demonstrated in particular that the organotoxicity of metal complexes or also other toxic mediums, like halogenated hydrocarbons and, in particular, both monohalogenated and polyhalogenated hydrocarbons, among these also the vapor anesthesias of the halothane type, as well as the corresponding aromatic hydrocarbons, nitrosamines, acrylamide or drugs, like paracetamol, methotrexate, isoniacide or aminoglycoride antibiotics or X-ray contrast mediums, can be suppressed. Therefore, the inventive drug is also suitable for the treatment of organotoxicity caused by environmental toxins, in particular as an antidote thereto, in organs, like the liver, kidney, central nervous system, pancreas, etc.

Hence, it also makes it possible, for example, to increase the dose of cytostatic drugs in the treatment of cancer and, against this background, may also raise, as an adjuvant therapy, the prospects of success in chemotherapy.

The pharmaceutical preparation of the invention is just as suited for treating acute renal failure, in particular such renal failures that are caused by drug intoxication, hemolytic disorders, the hemolytic uremic syndrome (Gasser's syndrome), rhabdomyolysis (necrosis of the striated skeletal muscles) by means of circulatory ischemic processes and/or are induced by a viral infection. In addition, this preparation has proven to be successful in the treatment of damages, which are caused by crushing the striated musculature (crush syndrome) and/or its necrosis on administration of prescription drugs (such as CSE inhibitors, for example, Lipobay).

It has proven to be quite especially suitable for preventing damage, resulting from the reperfusion of biological tissues, such as after an infarction of an organ, especially the heart, as well as the brain (cardiac infarction, stroke). Thus, for example, animal experiments have demonstrated that such reperfusion damage contributes from 60 to 80% of the tissue destruction and/or that the spread of tissue necrosis can be reduced by this factor. For a long time it has been known that reperfusion damage is caused predominantly by the oxygen radicals, which are formed during the ischemia.

Thus, the inventive preparation is also especially suitable for preventing reperfusion damage in transplanted organs. Such organs are kept in a cooled nutrient solution until they are transplanted into the body of a new recipient. Following the transplant, the body fluids flow through these organs, after being connected to the circulatory system of the recipient, as a result of which reperfusion damage occurs. An administration of the inventive preparation before and during the storage as well as just before the implant into the receiving organism may also solve this important transplant problem.

The inventive substances have proven to be successful, in particular, as inhibitors of human isoforms of the genetic family 2 of the cytochrome P450 and, in particular, the isoforms 2E1 and 2J2 and of the disorders, caused by them. An especially preferred embodiment of the invention provides that the pharmaceutical preparation be incorporated into the liposomes. Owing to the fact that the compounds, on which the preparation is based, exhibit long hydrocarbon radicals, their incorporation into liposomes is a very appropriate form of administration. Such liposomes are suitable for intravenous, intramuscular, intraperitoneal, percutaneous or also oral administration. An administration as an aerosol is just as suitable.

However, the inventive compounds may also be administered directly as such. In this case, too, the aforementioned types of administration are suitable.

### Methods of Synthesis

Several methods for synthesizing a wide array of inventive compounds are described below.

### 1. Synthesis of 12-imidizolyl-1-dodecanoic acid

a) 12-imidazolyl-1-dodecanoic acid is synthesized according to a method that is described in the article by Alterman et al. (“Fatty acid discrimination and omega-hydroxylation by cytochrome P450 4A1 and a cytochrome P4504A1/NADPH-P450 reductase fusion protein,” Archives of Biochemistry and Biophysics 1995, vol. 320, pp. 289-296).

To this end, 12-bromo-1-dodecanol is oxidized with Jones' reagent to form 12-bromo-1-dodecanoic acid. Then the white solid acid is esterified with diazomethane to form the corresponding methyl ester. The methyl ester is treated directly with imidazole and reacted at 80° C. for five hours until it forms 12-imidazolyl-1-dodecanoic acid methyl ester. The thick mass, which is obtained in this way, is separated into water and dichloromethane; and the organic phase is dried over Na₂SO₄ and concentrated by evaporation. The oily radical is cleaned chromatographically on silica gel and then dissolved in a mixture of methanol and tetrahydrofuran (3:4), treated with LiOH.H₂O, and the mixture is heated under reflux for two hours. Following evaporation of the solvent, the white residue is dissolved again in water, extracted with dichloromethane, acidified to a pH 5-6, and extracted again with ethyl acetate. The ethyl acetate extract is dried over Na₂SO₄, filtered and concentrated by evaporation. The white solid residue is recrystallized out of the methanol/ether and yields 12-imidazolyl-1-dodecanoic acid.

b) 12-imidazolyl-1-dodecanol and 1-imidazolyldodecane are synthesized according to a method that is described in the article by Lu et al. (“Heme-coordinating analogs of lauric acid as inhibitors of fatty acid cohydroxylation,” Archives of Biochemistry and Biophysics, 1997, Vol. 337, pp. 1-7). To this end, the temperature of 12-bromo-1-dodecanol and imidazole in a molar ratio of 1:3 is raised to 80° C. for five hours. The raw product is divided between water and dichloromethane. The organic phase is dried over Na₂SO₄ and concentrated by evaporation. The 12-imidazolyl-1-dodecanol is recrystallized out of benzene/n-hexane.

c) 1-imidazolyldodecane is produced from 1-bromododecane and imidazole in a molar ratio of 1:3 while stirring and raising the temperature to 85° C. The raw product is dissolved in dichloromethane and poured out three times with water. The organic phase is dried over Na₂SO₄, filtered and concentrated by evaporation. The oily evaporation residue is induced to crystallize from n-hexane and yields 1-imidazolyldodecane.

### 2. Synthesis of 12-imidazolyl-1-phosphatidylcholine

Phosphatidylcholine is reacted to form an O-phosphoryl thiourea under acidic conditions in the presence of dicyclohexylcarbodiimide. 12-imidazolyl-1-dodecanol is added to the reaction mixture. This 12-imidazolyl-1-dodecanol attacks nucleophilically the phosphoryl group and forms with this phosphoryl group an ester bond, so that 12-imidazolyl-1-phosphatidylcholine is formed. In so doing, dicyclohexylurea settles out. In order for this reaction to succeed, 4-diethylaminopyridine is necessary as the catalyst.

The reaction mechanism is similar to that of the Steglich esterification, where dicyclohexylcarbodiimide is used, in order to esterify an organic acid with an alcohol.

### 3. Synthesis of 1-palmitoyl-2-imidazolyl-glyerco-3-phosphatidylcholine

The principle for the synthesis of a phosphatidylcholine-digylceride, which carries an unmodified fatty acid and a labeled (that is, in the present case an ethinylated or imidazolized) fatty acid, is described by Eibl et al. (“Synthesis of labeled phospholipids in high yield,” Methods Enzymol. 1983, vol. 98, pp. 623-632).

### 3a. Synthesis of 1,2-dipalmitoyl-3-benzyl-glyceride

To this end, 1,2-isopropylidene-sn-glycerol is dissolved in p-xylene and stirred with the addition of potassium-tert-butylate and benzyl chloride. Upon completion of the reaction, water and diisopropyl ether are added in equal parts, and a phase separation is carried out. The 3-benzyl-sn-glycerol in the upper phase is obtained by evaporation and subjected to additional cleaning steps.

Then the cleaned 3-benzyl-sn-glycerol is dissolved with a fatty acid, for example palmitate, in carbon tetrachloride. With the addition of 4-diethylaminopyridine and dicyclohexylcarbodiimide, ester bonds are produced between the alcohol groups of the 3-benzyl-sn-glycerol and the carboxyl groups of the fatty acids, so that dicyclohexylurea settles out. This reaction mechanism is also called “Steglich esterification.”

The precipitated dicyclohexylurea is removed, and the solvent is removed by evaporation. Following additional cleaning steps, the product 1,2-dipalmitoyl-3-benzyl-sn-glycerol is obtained.

### 3b. Synthesis of 1,2-dipalmitoyl-sn-glyceride

1,2-dipalmitoyl-3-benzyl-sn-glyceride is dissolved in tetrahydrofuran and hydrogenolyzed with elementary hydrogen in the presence of a catalyst (10% Pd/C). In so doing, the benzyl radical is substituted with a hydrogen atom, and 1,2-dipalmitoyl-sn-glyceride is produced.

### 3c. Phosphorylation of 1,2-dipalmitoyl-sn-glyceride

Phosphoryl trichloride is treated with triethylamine, dissolved in tetrahydrofuran, and stirred in ice. Then 1,2-dipalmitoyl-sn-glyceride, dissolved drop-by-drop in tetrahydrofuran, is added. The result is then 1,2-dipalmitoyl-sn-glyceride-3-phosphoryl dichloride.

Then triethylamine, dissolved in tetrahydrofuran is added once more, bromoethanol, dissolved drop by drop in tetrahydrofuran, is added, and the temperature is raised to 25° C. The result is then predominantly 1,2-dipalmitoyl-sn-glyceride-3-phosphoryl-bromoethyl ester-monochloride and just a small quantity of the corresponding di-bromoethyl ester as a side product.

This mixture is cleaned, cooled, treated with sodium carbonate and hexane and shaken. In so doing, the bond between the phosphate radical and the chloride is hydrolyzed. The resulting product is the sodium salt of 1,2-dipalmitoyl-sn-glyceride-3-phosphoryl-bromoethyl ester.

The sodium salts of 1,2-dipalmitoyl-sn-glyceride-3-phosphoryl-(N-butoxycarbonyl)ethanolamine ester and 1,2-dipalmitoyl-sn-glyceride-3-phosphoryl-(N-butoxycarbonyl) tert-butyl serine ester are isolated in an analogous manner.

### 3d. Hydrolyzation of 1,2-dipalmitoyl-sn-glyceride-3-phosphoalkyl ester

1,2-dipalmitoyl-sn-glyceride-3-phosphoryl-bromoethyl ester or one of the aforementioned phosphoalkyl esters, which are presented as an alternative, is dissolved in a mixture of diethyl ether and distilled water that contains CaCl_{2.2}H₂O.

The pH is adjusted to 7.5 with the addition of a Palitzsch buffer. Then the enzyme phospholipase A₂ is added and stirred for 60 min. at 35° C. At the same time the ester bond at position 2 of the glycerol radical is hydrolyzed, and the resulting product is the corresponding 1-palmitoyl-sn-glyceride-3-phosphoalkyl ester, which carries an OH group at position 2, and a free fatty acid.

At this point the molecule that is obtained can be esterified specifically with a labeled fatty acid—for example, an imidazolized or ethinylated fatty acid—at position 2 of the glycerol radical. Similarly the phosphoalkyl ester can be re-esterified with a suitable alcohol—for example, choline, serine, ethanolamine or inositol—at position 3.

### 3e. Esterification with a labeled fatty acid at position 2

The obtained 1-palmitoyl-sn-glyceride-3-phosphoalkyl ester is dissolved in tetrachloromethane. An imidazolized or ethinylated fatty acid is added, and the mixture is stirred.

The fatty acid that is added may be, for example, 17-octadecinic acid, which is commercially available at Sigma Aldrich. Similarly it may be 12-imidazolyl-1-dodecanoic acid, which can be synthesized as described under 1.

Then a “Steglich esterification” is carried out once more; 4-diethylaminopyridine and dicyclohexylcarbodiimide are added to the mixture. At the same time an ester bond is formed between the remaining OH group at the glycerol radical and the carboxyl group of the labeled fatty acid.

The precipitated dicyclohexylurea is removed, and the solvent is removed by evaporation. Following additional cleaning steps, 1-palmitoyl-2-acyl-sn-glyceride-3-phosphoalkyl ester is obtained as the product.

### 3f. Re-esterification of the phosphoalkyl ester at position 3 of the glycerol radical

1-palmitoyl-2-acyl-sn-glyceride-3-phosphoryl-bromoethyl ester is dissolved in chloroform. Then 2-propanol-trimethylamine is added. The reaction vessel is incubated at 50° C. Then the solvent is evaporated with nitrogen. The reaction product is cleaned, and in this way a labeled 1-palmitoyl-2-acyl-sn-glyceride-3-phosphatidylcholine is obtained.

In order to isolate the labeled 1-palmitoyl-2-acyl-sn-glyceride-3-phosphatidyl-serine, the labeled 1-palmitoyl-2-acyl-sn-glyceride-3-phosphoryl-(N-butoxycarbonyl)ethanolamine ester, which is isolated as aforementioned, is dissolved in CH₂Cl₂, and trifluoroacetic acid and perchloric acid are added. Then the mixture is stirred in the cold state and washed with water and methanol. Following a phase separation, the lower phase is extracted with Na₂CO₃ and evaporated. Following the addition of methanol, crystals form. These crystals are the labeled 1-palmitoyl-2-acyl-sn-glyceride-3-phosphatidyl-ethanolamine.

A similar method is used to isolate labeled 1-palmitoyl-2-acyl-sn-glyceride-3-phosphatidylserine. In this case the parent substance is the labeled 1-palmitoyl-2-acyl-sn-glyceride-3-phosphoryl-(N-butoxycarbonyl) tert-butyl serine ester, which is isolated as aforementioned.

Tables

The attached tables list a few examples of the inventive compounds.

In this respect it is immediately clear to the person skilled in the art that a plurality of other compounds can be subsumed under the said claims. Thus, the aliphatic radicals may be straight chained or branched, exhibit single, double or triple bonds, and may be substituted, and exhibit an aliphatic backbone having 6 and/or 9 to 26 and/or 19 carbon atoms. Similarly the hydrocarbon backbone can be formed with alicyclic and/or aromatic hydrocarbons, so that in this case owing to the ring structures up to 40 carbon atoms may be necessary.

Suitable hydrophilic radicals are also other alcohols, like inositol or ethanolamine and/or their glycerides.

1.-10. (canceled)

11. A pharmaceutical composition comprising (a) a compound selected from the group consisting of: 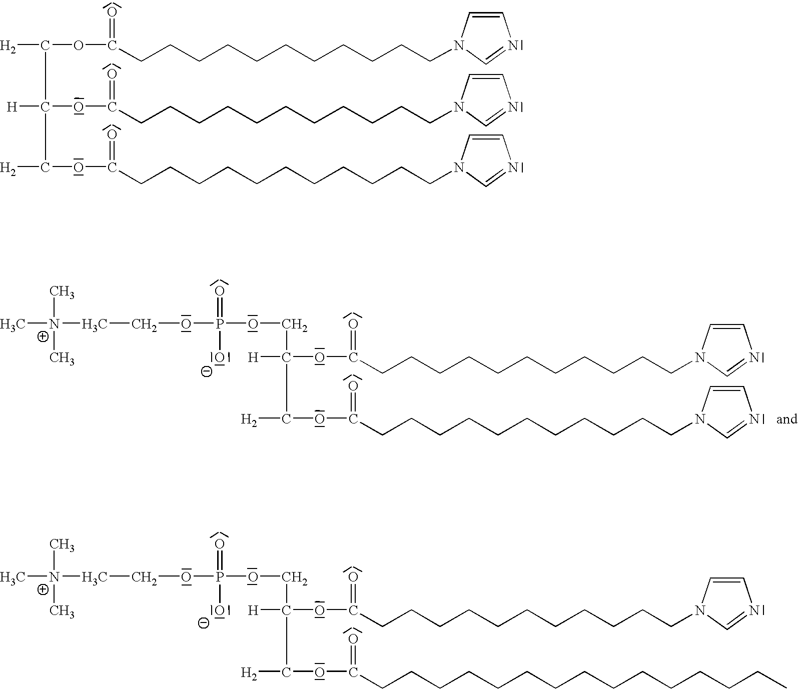
and pharmaceutically acceptable salts thereof, and (b) a pharmaceutically acceptable carrier.

12. The pharmaceutical composition according to claim 11, which is incorporated into liposomes.

13. A method for preventing or treating cancerous diseases, pathological sequelae of alcohol abuse, viral hepatitis, steatohepatitis, acute or chronic pancreatitis, toxic renal disorders, hepatic insulin resistance in diabetes mellitus, liver damage associated with Wilson' disease or sideroses, or for antidoting environmental toxins or prescription drug intoxication, or for prolonging the retention of drugs in the patient, or for combating toxic side effects on administration of chemotherapeutic agents, said method comprising administering to a patient in need thereof an effective amount therefor of a compound selected from the group consisting of: 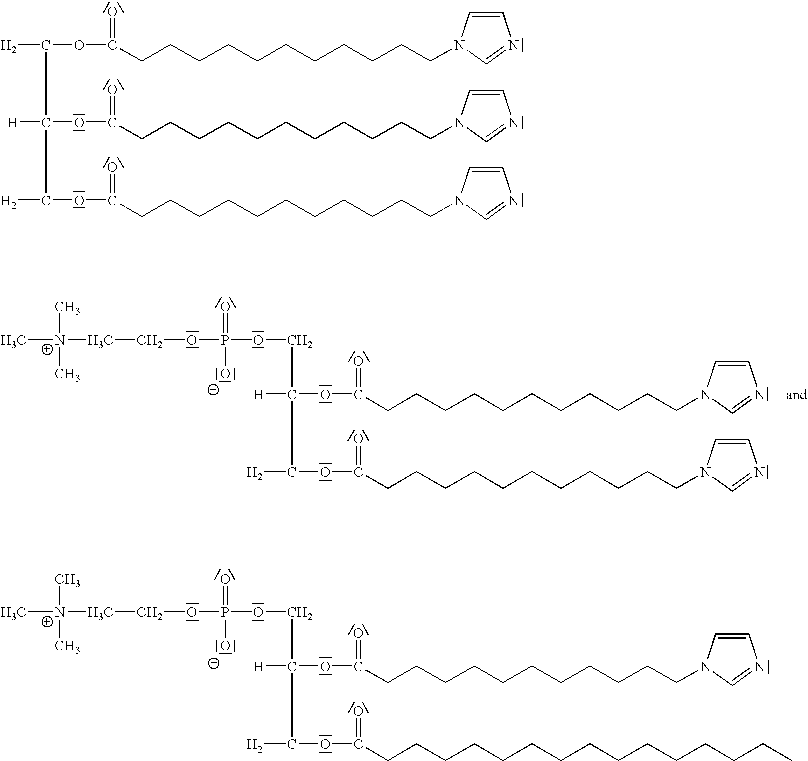
and pharmaceutically acceptable salts thereof.

14. The method according to claim 13, which is conducted for treating hyperlipidemia.

15. A method for preventing reperfusion damage in transplanted organs comprising administering to an organ to be transplanted to a patient or to the patient an effective amount therefor of a compound selected from the group consisting of: 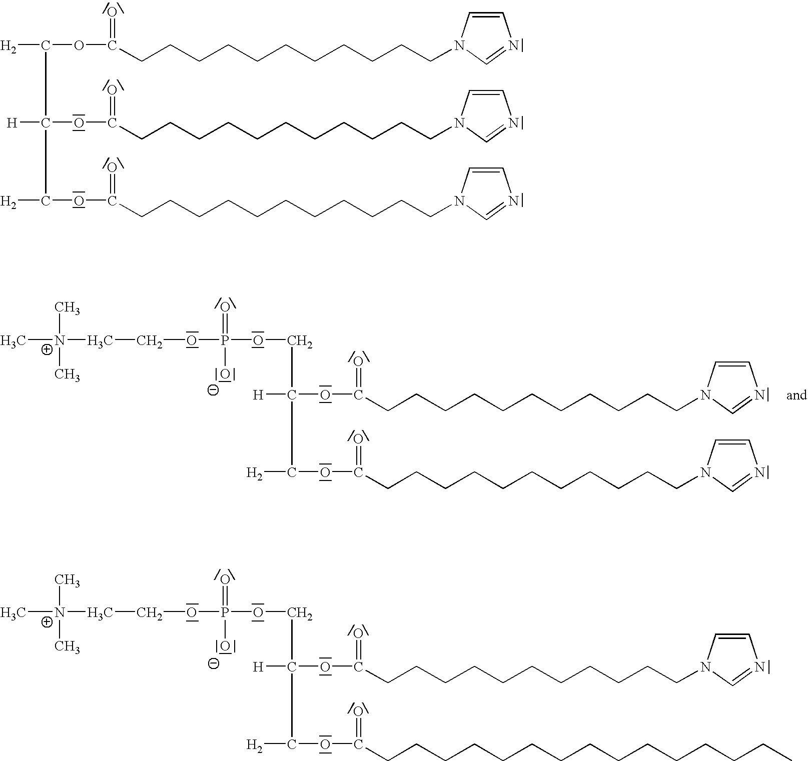
and pharmaceutically acceptable salts thereof.

16. The method according to claim 15, which comprises administering the compound or salt thereof to the organ to be transplanted before the organ is stored, while the organ is in storage or just prior to transplanting the organ to the patient.
